(19) United States
(12) Patent Application Publication
(10) Pub. No.: US 2008/0255521 A1
(43) Pub. Date: Oct. 16, 2008

(54) Prefilled syringe

(75) Inventors: Junichi Kubo, Tosu-shi (JP); Takaaki Yoshinaga, Tosu-shi (JP); Kazunori Muta, Tosu-shi (JP); Tetsuro Tateishi, Tosu-shi (JP)

Correspondence Address: NATH & ASSOCIATES, 112 South West Street, Alexandria, VA, 22314, US

(73) Assignee: HISAMITSU PHARMACEUTICAL CO. INC,, Tosu-shi (JP)

(21) Appl. No.: 12/081,246

(22) Filed: Apr. 11, 2008

(30) Foreign Application Priority Data
Apr. 12, 2007 (JP) P2007-105282

(51) Int. Cl. A61M 5/19

(52) U.S. Cl. 604/191

(57) ABSTRACT

In a prefilled syringe separately containing both a lidocaine solution and a hyaluronic acid solution, the viscosity of the hyaluronic acid solution is set so as to be sufficiently higher than the viscosity of the lidocaine solution. When lidocaine discharge from the tip of a hypodermic needle ends, the force f (N) required to discharge the hyaluronic acid solution from the needle tip suddenly increases, imparting sort of a jolt to the hand of the operator and creating a momentary sensation that the plunger is at rest. Letting the force f (N) required to discharge the lidocaine solution be P₁ and the force f (N) required to discharge the hyaluronic acid solution be P₂, the relationship therebetween is P₁<P₂. When (P₂−P₁)/P₂>0.2, the operator is able to feel a sufficient jolt.

### BACKGROUND OF THE INVENTION

1. Field of the Invention

The present invention relates to a syringe that is prefilled with a drug and the like.

2. Related Background Art

Injectable drugs, which are familiar as one type of drug dosage form, are sometimes supplied to the point of care as prefilled syringes. The prefilled syringe is a disposable syringe composed of a syringe barrel that also functions as a sealed container and is prefilled with a desired injectable drug. In recent years, considerable use has been made of such prefilled syringes because of the various advantages they provide. For example, they are easy and convenient to use, enabling a desired injectable drug to be correctly administered in the proper dose without error even in an emergency. Moreover, they are highly hygienic, making it possible to avoid bacterial and other microbial infections.

Examples of the construction of such prefilled syringes include the syringe construction disclosed in Japanese Patent Publication No. S62-58745 which enables one type of injectable drug to be prefilled in a hermetically sealed state within the syringe barrel. Japanese Patent Application Laid-open No. H8-308928 discloses a divided injection-type syringe construction wherein two different injectable drugs are separately prefilled into the syringe barrel in such a way as to enable the two injectable drugs to be sequentially injected.

A method used for treating various arthritic diseases such as osteoarthritis and chronic rheumatoid arthritis involves infusing a medication consisting of a hyaluronic acid solution at the site of the diseased joint in order to mitigate the impaired mobility and pain symptoms that arise from declines in the lubricating action of synovial fluid and its protective effects on the surface of arthrodial cartilage. Such an approach has shown a certain degree of effectiveness (see, for example, International Disclosure WO 2004/016275 and “Inflammation and Regeneration” (Ensho Saisei) Vol. 21, No. 6, p. 653 to 658 (November 2001), published by The Japanese Society of Inflammation and Regeneration).

Yet, it has been pointed out that when sodium hyaluronate is administered within the articular cavity at the affected site in the patient, or immediately following such administration, transient paint is experienced at the affected site. In this connection, Japanese Patent Application Laid-open No. 2003-299734 discloses, as a kit preparation for preventing severe pain in the affected area when an aqueous sodium hyaluronate solution is injected, a kit which uses a serial and sequential divided injection-type syringe containing an aqueous lidocaine solution as the first liquid medication and an aqueous sodium hyaluronate solution as a second liquid medication.

However, in spite of the desirability of a method of administration generally confirmed to be safe, i.e., administering an aqueous lidocaine solution outside of the articular cavity then administering an aqueous hyaluronic acid solution within the articular cavity after all the lidocaine solution has been completely administered outside of the cavity, using the serial and sequential divided injection-type syringe disclosed as noted above, one has no alternative but to visually check when all of the lidocaine solution has been fully administered. Such an approach thus leaves much to be desired in terms of convenience and ease of use.

### SUMMARY OF THE INVENTION

It is therefore an object of the present invention to provide a prefilled syringe for sequentially administering an aqueous lidocaine solution (first liquid medication) and an aqueous hyaluronic acid solution (second liquid medication), which prefilled syringe enables the liquid medications to be more safely and effectively administered.

As a result of extensive investigations, the inventors have discovered that the above object can be achieved by setting within specific ranges the viscosities of the liquid medications and the respective end stopper pushing forces when the liquid medication in a front chamber of the syringe barrel and the liquid medication in a back chamber of the syringe barrel are each discharged.

Accordingly, the prefilled syringe of the present invention is a prefilled double syringe having a barrel, an end stopper for sealing a first end of the barrel, a middle stopper interposed between the first end of the barrel and a second end of the barrel for dividing the interior of the barrel into a front chamber and a back chamber, a lidocaine solution enclosed within the front chamber, and a hyaluronic acid solution enclosed within the back chamber.

Here, the viscosity η_{LD} (mPas) of the lidocaine solution at 25° C., the mass percent concentration C_{LD} (wt %) of the lidocaine solution, the viscosity η_{HA} (mPas) of the hyaluronic acid solution at 25° C., the mass percent concentration C_{HA} (wt %) of the hyaluronic acid solution, the force P₁ (N) required to discharge the lidocaine solution enclosed within the front chamber from the second end of the barrel by pushing the end stopper toward the second end while causing the middle stopper to slide within the barrel and thereby applying dynamic frictional forces from an inner wall of the barrel to the end stopper and the middle stopper, and the force P₂ (N) required to discharge the hyaluronic acid solution enclosed within the back chamber from the second end of the barrel by pushing the end stopper toward the second end while applying a dynamic frictional force from the inner wall of the barrel to the end stopper are used as parameters.

These parameters satisfy the following conditions (A) to (C):

(A) 0.5≦η_{LD}≦5,

(B) 10≦η_{HA}≦600, and

(C) (P₂−P₁)/P₂>0.2.

In the present invention, because the viscosities have been set as noted above, when discharge of the lidocaine solution enclosed in the front chamber is complete, a distinctly larger force is required to discharge the hyaluronic acid solution enclosed in the back chamber. Given that the viscosity of the lidocaine solution is relatively low and the viscosity of the hyaluronic acid solution is relatively high, the hyaluronic acid solution in the back chamber cannot be easily discharged under the same pushing force. Therefore, once infusion of the lidocaine solution enclosed in the front chamber is complete, the hand of the operator immediately senses a jolt.

Moreover, when the pushing force difference ratio ((P₂-P₁)/P₂) exceeds 0.2 owing to regulation of the viscosities, the operator is able to feel a sufficient jolt. By setting the respective viscosities at or below specific values as noted above, the liquid medications can be discharged without any hindrance in flow. It should also be noted that pharmaceutically effective lidocaine solutions have a viscosity of 0.5 mPas or more.

Although each of the pushing forces P₁ and P₂ required to discharge the solutions vary somewhat depending on the stroke length of the end stopper, these forces P₁ and P₂ are assumed to be given by the average values of the forces P₁ and P₂ required when the stoppers move within the respective corresponding stroke ranges while dynamic frictional forces are at work.

Therefore, when an injection to the articular cavity is carried out, for example, the operator is able, by means of the jolt that is sensed, to know when infusion of the lidocaine solution outside of the articular cavity is complete, and can then easily inject the hyaluronic solution into the articular cavity. It is thus possible to more safely and effectively administer the liquid medications. Of course, the syringe of the present invention is not limited to use only for administering medication to the articular cavity; it may be similarly employed in other instances where hyaluronic acid is used as an adjuvant or eye medication and lidocaine solution is used at the same time as a local anesthetic, including procedures such as intraocular lens implantation and full-thickness corneal grafting.

The lidocaine solution is a solution which contains lidocaine as the main ingredient, and may also contain other ingredients provided they do not compromise the pharmaceutical effects of the lidocaine solution. Here, “lidocaine solution” also encompasses solutions of hydrochloride acid salts of lidocaine such as lidocaine hydrochloride. Lidocaine is a substance which has the pharmacological action of blocking nerve transmission by inhibiting the passage of sodium ions and thus inactivating the action potential.

The hyaluronic acid solution is a solution which contains hyaluronic acid, and may also contain other ingredients provided they do not compromise the pharmaceutical effects of the hyaluronic acid solution. Here, “hyaluronic acid solution” also encompasses solutions of sodium salts of hyaluronic acid such as sodium hyaluronate. Sodium hyaluronate is known as a therapeutic agent for various diseases of the joints and as a humectant.

The viscosity of a liquid medication is closely connected to its concentration, the tendency being for the viscosity to increase at higher concentrations. Moreover, the more dilute such a solution is, the weaker its pharmaceutical effects tend to be. Therefore, to achieve the above-indicated difference ratio in the viscosities and thus reliably impart a jolt to the operator while retaining the pharmaceutical effects of the liquid medications, it is preferable to satisfy the following conditions (D) and (E).

(D) 0.5≦C_{LD}≦2, and

(E) 0.5≦C_{HA}≦2.

In addition, the above-described parameters preferably satisfy the following conditions (F) and (G).

(F) P₂−P₁>2, and

(G) P₂<40.

When above condition (F) is satisfied, the difference between the respective pushing forces exceeds 2N, enabling the operator to more reliably sense a jolt. However, if the pushing forces required are too high, the act of injection itself cannot be smoothly carried out. Hence, the pushing force P₂ (N) required to discharge the hyaluronic acid solution enclosed in the back chamber is set to below 40 N so as to enable injection to be smoothly carried out.

The present invention thus provides a prefilled syringe which enables liquid medications to be more safely and effectively administered.

### BRIEF DESCRIPTION OF THE DRAWINGS

FIG. 1 is a perspective view of a prefilled syringe;

FIG. 2 is longisectional view of the syringe shown in FIG. 1 at the time of use;

FIG. 3 is a sectional view of the syringe in FIG. 2, taken along III-III;

FIG. 4 is a graph which schematically shows the relationship between the stroke (arbitrary units) of a plunger 12 having an end stopper 12A and the pushing force f(N) applied to the end stopper 12A;

FIG. 5 is a table showing the types and amounts (mg) of ingredients in a first liquid medication A;

FIG. 6 is a table showing the types of amounts (mg) of ingredients in a second liquid medication B;

FIG. 7 is a table showing the measured pushing forces P₁ and P₂, the pushing force difference P₂−P₁ and the difference ratio (P₂−P₁)/P₂ for prefilled syringes in Specimens No. (1) to (6) using hyaluronic acid solutions having different viscosities μ_{HA} (mPas) and mass percent concentrations C_{HA} (wt %);

FIG. 8 is a graph of stroke (mm) versus force f (N) in Specimen No. (1);

FIG. 9 is a graph of stroke (mm) versus force f (N) in Specimen No. (2);

FIG. 10 is a graph of stroke (mm) versus force f (N) in Specimen No. (3);

FIG. 11 is a graph of stroke (mm) versus force f (N) in Specimen No. (4);

FIG. 12 is a graph of stroke (mm) versus force f (N) in Specimen No. (5);

FIG. 13 is a graph of stroke (mm) versus force f (N) in Specimen No. (6); and

FIG. 14 is a table showing the results obtained when four different operators A to D pushed the plunger.

### DESCRIPTION OF THE PREFERRED EMBODIMENTS

Preferred embodiments of the prefilled syringe of the invention are described below. In the description that follows, like elements are denoted by like reference symbols and the unnecessary repetition of explanations is avoided.

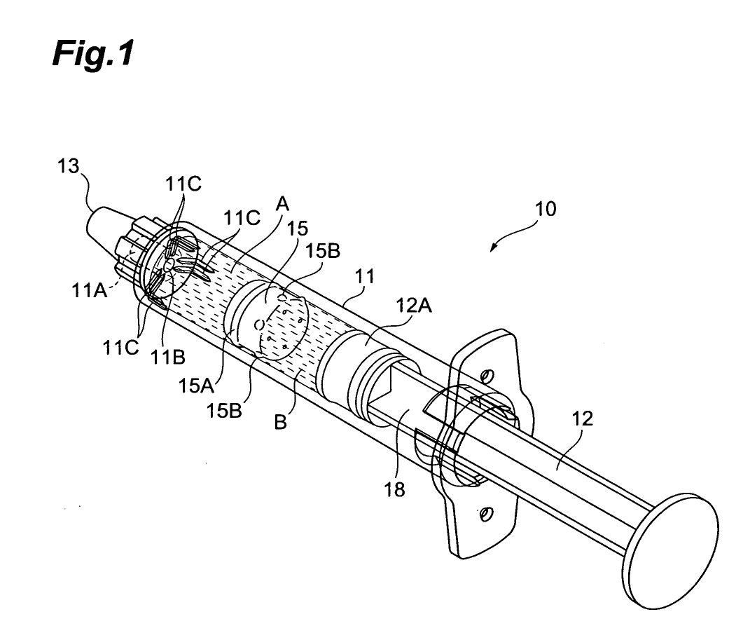

FIG. 1 is a perspective view of a prefilled syringe.

A double syringe-type prefilled syringe 10 according to the present embodiment has a barrel 11, a plunger 12 with an end stopper 12A for sealing a first end of the barrel 11, a middle stopper 15 interposed between the first end and a second end of the barrel 11 for dividing the interior of the barrel 11 into a front chamber and a back chamber, a first liquid medication (lidocaine solution) A enclosed within the front chamber, and a second liquid medication (hyaluronic acid solution) B enclosed within the back chamber. The end stopper 12A and the middle stopper 15 slide over an inner peripheral wall of the barrel 11.

The plunger 12 includes a piston body 18 to the end of which the end stopper 12A is screwed or otherwise attached. In a disposable syringe, the barrel and piston are generally made of polypropylene, poly(4-methylpentene-1) or the like. However, in the syringe 10 according to the present embodiment, the barrel 11 and the piston body 18 are formed of a cyclic olefinic polymer (COP) resin which is heat resistant and non-staining.

COP resins can be broadly divided into the following two types.

In a first type, the COP resins are copolymers of (a) a cyclic olefin and (b) an acyclic olefin.

In a second type, the COP resins are cyclic olefin ring-opening metathesis polymers (products prepared by the ring-opening metathesis polymerization of a cyclic olefin (a)) or cyclic olefin ring-opening metathesis polymer hydrogenates (products obtained by the ring-opening metathesis polymerization of a cyclic olefin (a), followed by hydrogenation of the polymer).

Illustrative examples of cyclic olefins (a) include polycyclic olefins having a norbomene ring (e.g., norbomenes, dicyclopentadienes, tetracyclododecenes), monocyclic olefins and cyclic diolefins.

Illustrative examples of acyclic olefins (b) include vinyl group-bearing compounds (α-olefins) and (meth)acryloyl group-bearing compounds.

Of the above, the COP resin in the present embodiment is most preferably a cyclic olefin ring-opening metathesis polymer hydrogenate.

To maintain airtightness, the end stopper 12A is often formed of an elastic material such as rubber or a thermoplastic elastomer.

Examples of suitable rubbers include, but are not particularly limited to, compounds which contain as the primary starting material a synthetic rubber such as isoprene rubber, butadiene rubber, styrene-butadiene rubber, ethylene-propylene rubber, isoprene-isobutylene rubber or nitrile rubber, or a natural rubber, and in which other ingredients such as fillers and crosslinking agents have been incorporated.

Thermoplastic elastomers that may be used include solution polymerization type styrene-butadiene rubbers (e.g., SBS bock copolymers), polyester or polyether urethane rubbers, polyether aromatic polyester block copolymers (polyester rubbers), polyolefin block copolymers, high trans-1,4-polyisoprene, polyethylene-butyl graft copolymers and syndiotactic polybutadiene.

In addition to the above, relatively soft plastics, such as copolymer-type polypropylene, low-density polyethylene, ethylene-vinyl acetate copolymers and other copolymer-type plastics which have about the same degree of heat resistance (preferably about 130 to 140° C.) as polypropylene may be used.

The double syringe-type prefilled syringe 10 has a barrel 11 and a plunger 12, which slidably inserts into the barrel 11, having an end stopper 12A, and disposed between the end stopper 12A and the tip of the barrel 11, a middle stopper 15 which defines and forms within the barrel 11 a front chamber and back chamber.

The middle stopper 15 is made of, for example, a disc-shaped rubbery elastic body having a front end on an outer periphery of which an elastically deformable ring-shaped lip 15A is formed so as to be in sliding contact with the inner peripheral wall of the barrel 11. The middle stopper 15 has a back end on an outer periphery of which a plurality of guide projections 15B are formed so as to be in sliding contact with the inner peripheral wall of the barrel 11 and to prevent, in concert with the lip 15A, the axis of the middle stopper 15 from tilting.

To maintain airtightness, the middle stopper 15 is often formed of an elastic material such as a rubber or thermoplastic elastomer. Examples of the rubber include, but are not particularly limited to, compounds which contain as the primary starting material a synthetic rubber such as isoprene rubber, butadiene rubber, styrene-butadiene rubber, ethylene-propylene rubber, isoprene-isobutylene rubbers or nitrile rubber, or a natural rubber, and in which other ingredients such as fillers and crosslinking agents have been incorporated.

The barrel 11 has formed, on an inner face at the tip thereof, three pairs of bypass-forming projections 11C which radially extend from the periphery of an injection port 11B in a needle mount 11A toward the inner peripheral wall of the barrel 11 at equiangular intervals.

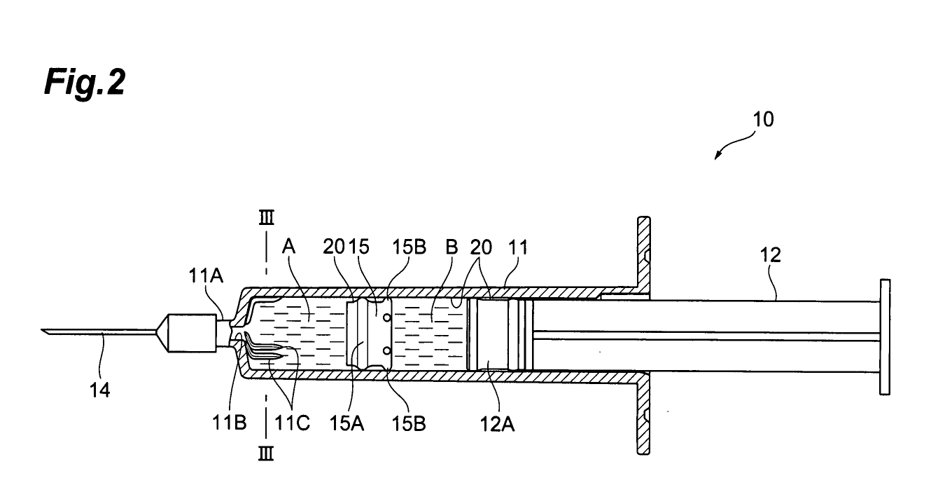

FIG. 2 is a longisectional view of the syringe 10 shown in FIG. 1 at the time of use. When the prefilled syringe 10 is not in use, a top cap 13 (see FIG. 1) for hermetic sealing is attached to the small-diameter needle mount 11A formed at the tip of the barrel 11, at the time of use of the prefilled syringe 10 from which the top cap 13 has been removed, a hypodermic needle 14 is mounted onto the needle mount 11A in place of the top cap 13. Hypodermic needles 14 having a size of from 22 G to 23 G (a bore diameter of from 0.48 mm to 0.40 mm) are commonly used at the point of care.

The inner peripheral wall of the barrel 11, the surface of the end stopper 12A and the surface of the middle stopper 15 may each be coated with a silicone gel layer. Silicones for forming the silicone gel layer 20 may be broadly categorized, according to the type of organic groups bonded to the silicon atoms, into (a) straight silicones and (b) modified silicones.

Straight silicones (a) are silicones in which methyl groups and hydrogen atoms are bonded as substituents.

Modified silicones (b) are silicones which have structural portions secondarily derived from straight silicone. Examples include organopolysiloxanes having at least one (and preferably at least two) unsaturated group such as a vinyl group or a (meth)acryloyl group.

In the present embodiment, the silicone is not limited to the forgoing silicones, and may be either a straight silicone or a modified silicone. For example, use may be made of Dow Corning 360 (manufactured by Dow Corning), which is a known straight silicone that cures on exposure to gamma rays, or Three Bond 3167 or 3168 (manufactured by Three Bond), which are commercially sold as ultraviolet-curable modified silicone gels. The use here of Dow Corning 360 (manufactured by Dow Corning) is most preferred.

In the syringe 10, by providing a silicone gel layer 20 on the inner peripheral wall of the barrel 11 and/or the surfaces of the stoppers (the end stopper 12A and the middle stopper 15), slidability between the barrel 11 and the stoppers can be ensured. Moreover, the risk of silicone separation and delamination over time from the plastic of the barrel 11 can be reduced, enabling liquids to be more stably held within the barrel 11.

When the plunger 12 having an end stopper 12A is pushed in the direction of the hypodermic needle 14, the second liquid medication B presses against the middle stopper 15, and the middle stopper 15 being pressed on in turn presses against the first liquid medication A, causing the first liquid medication A to flow out to the exterior through the injection port 11B and the hypodermic needle 14. When the first liquid medication A finishes flowing out, the projections 11C deform the outer peripheral edge of the middle stopper 15, forming bypass channels. Additionally pushing the plunger 12 having an end stopper 12A in the direction of the hypodermic needle 14 causes the end stopper 12A to press against the second liquid medication B, causing the second liquid medication B to flow out to the exterior through the bypass channels, injection port 11B and hypodermic needle 14.

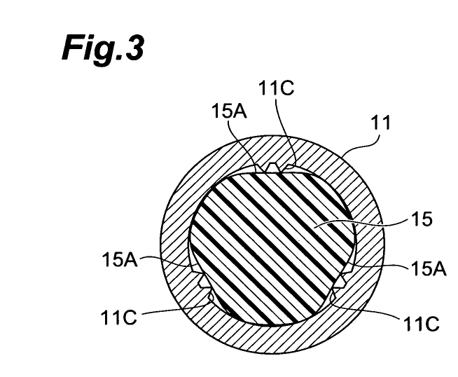

FIG. 3 is a sectional view along III-III of the syringe shown in FIG. 2. When the middle stopper 15 moves toward the tip of the barrel 11, the three pairs of bypass-forming projections 11C shown in FIGS. 1 and 2 come into contact with the outer peripheral edge of the middle stopper 15, as a result of which the portions of the projections 11C that extend out from the inner peripheral wall of the barrel 11 elastically deform the lip 15A of the middle stopper 15, forming bypass channels which communicate between the back chamber behind the middle stopper 15 and the injection port 11B.

When the second liquid medication B having a high viscosity passes through the bypass channels, it creates eddies near the bypass channel outlets, elevating the pushing force P₂. In the cross-section taken perpendicular to the lengthwise direction of the barrel 11 shown in FIG. 3, the cross-sectional surface area S₁₁, of the opening defined by the inside diameter of the barrel 11 is 120 mm²in the present embodiment. The total cross-sectional surface area of the bypass channels formed as gaps between the outer peripheral edge of the middle stopper 15 and the inner wall of the barrel 11 is designated herein as S_{BT}. Letting the cross-sectional surface area of one cross-sectionally trapezoidal bypass channel formed by neighboring projections 11C be S_{B}, given that cross-sectionally trapezoidal bypass channels are formed in three places in the present embodiment, the total cross-sectional surface area for the bypass channels is 3×S_{B}. In the construction shown in FIG. 3, specifying the cross-sectional surface area S_{B} has the effect of defining the total cross-sectional surface area S_{BT} of the bypass channels.

To generate the above-described jolt, the total cross-sectional surface area S_{BT} of the bypass channels is preferably not more than 10% (12 mm²), more preferably not more than 8% (9.6 mm²), and even more preferably not more than 3% (3.6 mm²) of the cross-sectional surface area S₁₁ of the barrel opening.

Alternatively, to generate the above-described jolt, it is preferable for the cross-sectional surface area S_{B} of the individual bypass channels to be 1 mm² or less, and for the total cross-sectional surface area S_{BT} of the bypass openings to be 5 mm² or less.

If the bypass channels are too narrow, smooth flow of the liquid medication cannot be carried out. Accordingly, it is preferable for the cross-sectional surface area S_{B} of the individual bypass channels to be at least 0.03 μm² and for the minimum inside diameter of the bypass channels to be at least 0.2 μm. A cross-sectional surface area S_{B} of at least 0.1 mm² is more preferred.

To smoothly carry out the flow of liquid medication, the total cross-sectional surface area S_{BT} of the bypass channels is preferably at least 0.03 μm², and more preferably at least 1 mm².

In the prefilled syringe 10 constituted as described above, the front chamber formed in front of the middle stopper 15 within the barrel 11 is prefilled with a predetermined amount of lidocaine solution as the first liquid medication A. The back chamber formed in back of the middle stopper 15 within the barrel 11 is prefilled with a predetermined amount of hyaluronic acid solution as the second liquid medication B.

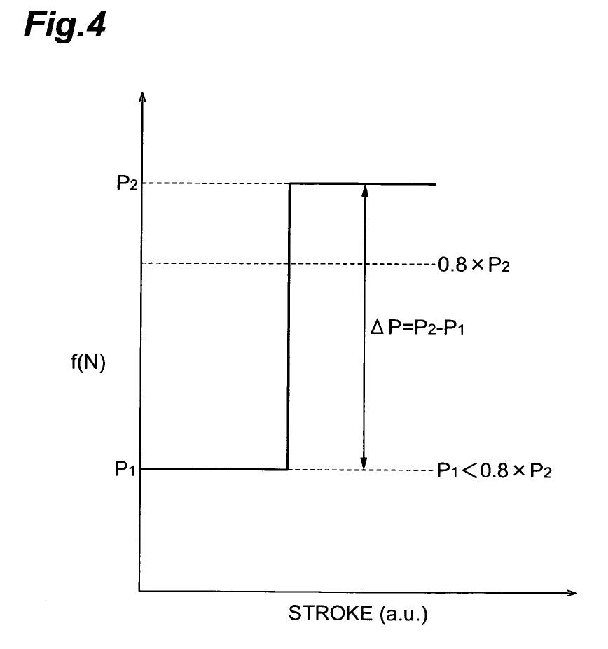

FIG. 4 is a graph which schematically shows the relationship between the stroke length (arbitrary units) of the plunger 12 having an end stopper 12A and the force f (N) pushing the end stopper 12A. Because the plunger 12 and the end stopper 12A are mechanically coupled, the force f (N) pushing on each and the stroke length of each are identical for both.

The larger the stroke of the end stopper 12A, the closer the end stopper 12A approaches to the tip (injection port 11B) of the barrel 11. The end stopper 12A pushes the second liquid medication B, the second liquid medication B pushes the middle stopper 15, the middle stopper 15 pushes the first liquid medication A, and the first liquid medication A is discharged from the tip of the hypodermic needle 14. When the end stopper 12A is pushed, large static frictional forces initially act between the respective stoppers 12A and 15 and the inner wall of the barrel 11. However, because such forces are not directly related to the principle of operation in the present invention, they are not shown in the diagram. When the respective stoppers 12A and 15 begin to move, dynamic frictional forces act between the respective stoppers 12A and 15 and the inner wall of the barrel 11, and the stoppers 12A and 15 are pushed toward the tip of the barrel 11 against pressure from the respective liquid medications and against dynamic frictional forces from the inner wall of the barrel 11.

The dynamic frictional forces are substantially constant and relatively small. Hence, the force required to push the stoppers 12A and 15 depends on the pressure from the liquid medications. The pressure from the liquid medications is governed by the viscosity of the liquid medication that flows out of the hypodermic needle 14.

When the viscosity of the second liquid medication B is set so as to be sufficiently higher than the viscosity of the first liquid medication A and discharge of the first liquid medication A comes to an end, the force f (N) required to cause the second liquid medication B to flow out the tip of the hypodermic needle 14 suddenly increases, imparting sort of a jolt to the hand of the operator; that is, giving rise to a momentary sensation that the plunger 12 is at rest. Here, letting the force f (N) required to discharge the first liquid medication A while the above dynamic frictional forces are at work be P₁ and letting the force f (N) required to discharge the second liquid medication B while the dynamic frictional forces are at work be P₂, P₁<P₂.

The difference between these pushing forces (ΔP=P₂−P₁) is made larger than 2 (N), enabling the operator to easily detect when discharge of the first liquid medication A ends and discharge of the second liquid medication B begins. Moreover, P₁ is smaller than 0.8×P₂ (P₁<0.8×P₂), making it possible to clearly sense the difference in the relative pushing forces. That is, when the condition P₁<0.8×P₂ is satisfied, the relationship (P₂−P₁)/P₂>0.2 holds. In such a case, the operator is able to fully sense a jolt.

The pushing force parameters for the first liquid medication (lidocaine solution) A and the second liquid medication (hyaluronic acid solution) B are defined as follows.
η_{LD} (mPas): Viscosity of lidocaine solution at 25° C.
C_{LD} (wt %): mass percent concentration of lidocaine solution
η_{HA} (mPas): Viscosity of hyaluronic acid solution at 25° C.
C_{HA} (wt %): Mass percent concentration of hyaluronic acid solution
P₁ (N): Force required to discharge the lidocaine solution enclosed within the front chamber from the tip of the barrel 11 by pushing the end stopper 12A toward the tip of the barrel 11 while causing the middle stopper 15 to slide within the barrel 11 and thereby applying dynamic frictional forces from an inner wall of the barrel 11 to the end stopper 12A and the middle stopper 15
P₂ (N): Force required to discharge the hyaluronic acid solution enclosed within the back chamber from the tip of the barrel 11 by pushing the end stopper 12A toward the tip of the barrel 11 while applying a dynamic frictional force from the inner wall of the barrel 11 to the end stopper 12A

The present embodiment satisfies the following conditions:

(A) 0.5≦η_{LD}≦5

(B) 10≦η_{HA}≦600, and

(C) (P₂−P₁)/P₂>0.2.

Here, because the viscosities are set as indicated above, when discharge of the lidocaine solution enclosed in the front chamber comes to an end, a distinctly larger force is required to discharge the hyaluronic acid solution enclosed in the back chamber. The reason is that, because the lidocaine solution has a relatively low viscosity and the hyaluronic acid solution has a relatively high viscosity, the hyaluronic acid solution in the back chamber cannot be readily discharged under the same pushing force. Therefore, immediately after infusion of the lidocaine solution in the front chamber comes to an end, the hand of the operator senses a jolt.

By regulating the viscosities so that the difference ratio for the pushing forces ((P₂−P₁)/P₂) is greater than 0.2, it is possible for the operator to adequately sense a jolt. Because, as noted above, the viscosities of the respective liquid medications have been set at or below given values, the liquid medications can be discharged without hindrance to the flows thereof. It should also be noted that pharmaceutically effective lidocaine solutions have a viscosity η_{LD} of at least 0.5 mPas. For the impact when discharging the hyaluronic acid solution in the back chamber to be sensed even more strongly, it is preferable for the viscosity η_{LD} of the lidocaine solution in the front chamber to satisfy the condition 0.5≦η_{LD}≦2.

Although each of the pushing forces P₁ and P₂ required to discharge the solutions vary somewhat depending on the stroke length of the end stopper 12A, these forces P₁ and P₂ are assumed to be given by the average values of the forces P₁ and P₂ required when the stoppers 12A and 15 move within the respective corresponding stroke ranges while dynamic frictional forces are at work. The dynamic frictional forces of the stoppers 12A and 15 are sufficiently smaller than the resistance forces due to flow friction when the liquid medications flow out of the hypodermic needle 14.

In the present invention, when an injection to the articular cavity is carried out, the operator is able, by means of the jolt that is sensed, to known when infusion of the lidocaine solution outside of the articular cavity is complete, and can then easily inject the hyaluronic acid solution into the articular cavity. It is thus possible to more safely and effectively administer the liquid medications. Of course, the above-described syringe 10 is not limited to use only for administering medication to the articular cavity; it may be similarly employed in other instances where hyaluronic acid is used as an adjuvant or eye medication and lidocaine solution is used at the same time as a local anesthetic, including procedures such as intraocular lens implantation and full-thickness corneal grafting.

The viscosities of the liquid medications are closely connected to their concentrations, with the viscosities tending to be higher at higher concentrations. Moreover, the more dilute the solution, the weaker its pharmaceutical effects tend to be. Therefore, to achieve the above-indicated viscosity difference ratio ((P₂−P₁)/P₂ >0.2) and thus reliably impart a jolt to the operator while retaining the pharmaceutical effects of the liquid medications, it is preferable to satisfy the following conditions (D) and (E):

(D) 0.5≦C_{LD}≦2, and

(E) 0.5≦C_{HA}≦2.

In addition, the above parameters preferably satisfy the following conditions (F) and (G):

(F)P₂−P₁>2, and

(G) P₂<40.

When condition (F) is satisfied, the difference between the respective pushing forces exceeds 2N, enabling the operator to more reliably sense a jolt. However, when the pushing forces required are too high, the act of injection itself cannot be smoothly carried out. Hence, the pushing force P₂ (N) required to discharge the hyaluronic acid solution enclosed in the back chamber is set to below 40 N so as to enable injection to be smoothly carried out without the application of unnecessary force by the hand operating the syringe.

Next, the first liquid medication A and the second liquid medication B are described more fully.

The first liquid medication A is a 0.5 to 2 wt % lidocaine solution. Although this solution is not subject to any particular limitation, in the present embodiment it is an aqueous solution having a composition which includes, in addition to lidocaine, other ingredients such as sodium chloride, hydrochloric acid, sodium hydroxide and distilled water for injection. This aqueous lidocaine solution has a viscosity of from 0.5 mPas to 5 mPas. The viscosity is a value obtained by measuring a sample at room temperature (25° C.) using a digital Brookfield type viscometer (model RVDV-III, manufactured by Brookfield).

The lidocaine solution, so long as it is a solution containing lidocaine as the main ingredient, may include other ingredients provided the pharmaceutical effects are not thereby compromised, and encompasses solutions of hydrochloric acid salts of lidocaine such as lidocaine hydrochloride. Lidocaine is known to be a substance which has the pharmacological action of blocking nerve transmission by inhibiting the passage of sodium ions and thus inactivating the action potential. The lidocaine solution contains lidocaine and/or a pharmacologically acceptable salt thereof as a local anesthetic. It is preferable for the pH of the lidocaine solution to be adjusted in a range of from 5.0 to 7.0.

The second liquid medication B is a 0.5 to 2 wt % hyaluronic acid solution. So long as it is a solution which includes hyaluronic acid, the hyaluronic acid solution may also contain other ingredients to the extent that such additional ingredients do not compromise the pharmaceutical effects of the solution. The present embodiment uses a 1 wt % solution of hyaluronic acid. This solution contains sodium hyaluronate as an agent for treating various arthritic diseases such as osteoarthritis and chronic rheumatoid arthritis. The ingredients included in the hyaluronic acid solution of the present embodiment are sodium hyaluronate, sodium chloride, monobasic sodium phosphate, dibasic sodium phosphate, sodium hydroxide, hydrochloric acid and distilled water for injection. It is preferable for the pH of the hyaluronic acid solution to be adjusted in a range of from 6.8 to 7.8. Sodium hyaluronate is also known as a therapeutic agent for various diseases of the joints and as a humectant.

The viscosity of the hyaluronic acid solution varies considerably with adjustments in the concentration. The viscosity is also dependent on the molecular weight. The hyaluronic acid solution in the present embodiment is a sodium hyaluronate solution. The sodium hyaluronate included in the solution may be one having a molecular weight of from 600,000 to 4,000,000, and is preferably one having a molecular weight of from 600,000 to 1,200,000. Owing to the viscosity-based flow frictional forces of the liquid medication, and also to set the pushing forces P₁ and P₂ within the above-indicated ranges, it is preferable for the viscosity of the aqueous solution of sodium hyaluronate to be set within a range of from 10 mPas to 600 mPas.

In the above-described syringe, by thus setting the viscosities of the first liquid medication (lidocaine solution) A within the front chamber and the second liquid medication (hyaluronic acid solution) B within the back chamber to predetermined viscosities, the forces required to discharge the liquid medications through the hypodermic needle 14 can be adjusted to specific ranges, enabling the plunger to impart to the hand of the operator the sensation that the plunger has momentarily stopped following completion of the administration of the lidocaine solution.

Moreover, by satisfying all of the above conditions, jolt is more reliably imparted to the hand of the operator holding the syringe during administration of the injection to a patient, thus enabling the operator to know when administration of the first liquid medication A is complete. The operator, after sensing such a jolt, is then able to administer the second liquid medication B (sodium hyaluronate) into the articular cavity without withdrawing the hypodermic needle and without having to visually check the syringe.

Next, sterilization treatment is explained.

Ordinarily, when a liquid medication for injection is sterilized, high-pressure steam sterilization is carried out at the liquid medication stage or following assembly with the injection device. However, in such sterilization, sodium hyaluronate decomposes and its molecular weight decreases, as a result of which the viscosity of the aqueous solution of sodium hyaluronate may decline.

A method of sterilization which either leaves the molecular weight of the sodium hyaluronate substantially unaltered or, even if it does alter the molecular weight, has substantially no effect on the viscosity of the sodium hyaluronate solution can be employed in sterilization treatment in the manufacture of the above-described syringe. Such sterilization methods may be suitably selected from among, for example, high-pressure steam sterilization and filtration sterilization. Of these, to avoid a change in the molecular weight of the sodium hyaluronate, it is more preferable to use the subsequently described filtration sterilization because an aqueous solution of sodium hyaluronate having a desired viscosity of between 10 and 600 mPas can be obtained with substantially no decrease in viscosity.

The method of filtration sterilization is not subject to any particular limitation, provided the above-indicated hyaluronic acid solution (e.g., aqueous sodium of sodium hyaluronate) viscosity can be obtained. However, it is preferable to carry out filtration at a temperature of from 40 to 80° C. and a pressure of from 100 to 500 kPa using a membrane filter which is made of hydrophilic polyethersulfone or hydrophilic polyvinylidene difluoride and has a pore size of 0.2 μm.

When the filtration sterilization temperature is set to from 40 to 80° C., the decomposition that is a concern in high-pressure steam sterilization either does not arise or substantially does not arise. A filtration sterilization temperature below 30° C. is undesirable because the filtration rate shows a tendency to decline and clogging of the filer may arise. On the other hand, a filtration sterilization temperature above 80° C. is undesirable because the hyaluronic acid shows a tendency to decompose.

The filtration pressure is from 100 to 500 kPa, and preferably from 300 to 350 kPa. At a filtration pressure below 100 kPa, the filtration rate is slow, which is undesirable in terms of the efficiency of the operation. A filtration pressure above 500 kPa is undesirable because a sufficient filtration effect tends to be unobtainable.

The filter used for filtration is made of hydrophilic polyethersulfone or hydrophilic polyvinylidene difluoride and has a pore size of 0.2 μm. Using such a filter, even a viscous hyaluronic acid solution can be filtered without clogging of the pores. An aqueous sodium hyaluronate solution of the above-indicated viscosity can be efficiently obtained by using most preferably a filter composed of a hydrophilic polyethersulfone.

### EXAMPLES

In the examples described below, the prefilled syringe 10 was one in which the barrel 11 was made of a COP resin, the end stopper 12A and the middle stopper 15 were made of rubber, and each of these components was coated with a silicone gel layer. The bypass channels in the construction shown in FIG. 3 each had a cross-sectional surface area S_{B} of 0.5 mm².

The front chamber of the prefilled syringe 10 in the above-described embodiment was filled with an aqueous solution of lidocaine as the first liquid medication A, and the back chamber was filled with an aqueous solution of sodium hyaluronate as the second liquid medication B.

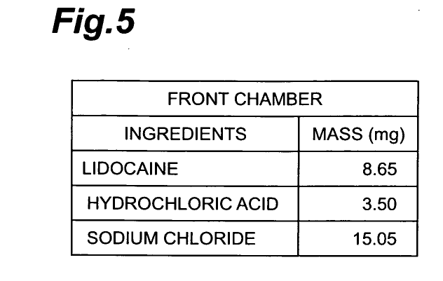

FIG. 5 shows the ingredients and mass (mg) of the first liquid medication A. The first liquid medication A was composed of 2.0 ml of a 0.5 wt % lidocaine solution (viscosity, 0.9 mPas).

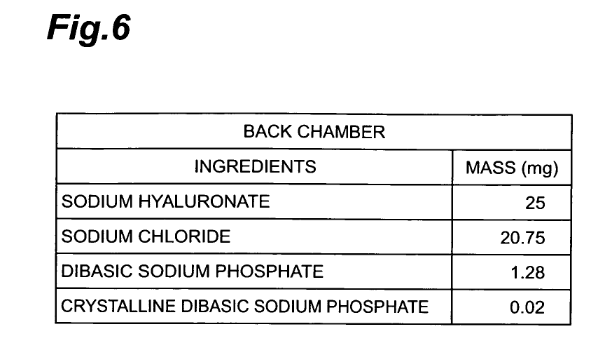

FIG. 6 shows the ingredients and mass (mg) of the second liquid medication B. The second liquid medication B was composed of 2.5 ml of a sodium hyaluronate solution having a viscosity and a mass percent concentration CHA (wt %) different from those of the first liquid medication A.

Each of the liquid medications contained distilled water for injection as the solvent and was pH adjusted to substantial neutrality by the addition of hydrochloric acid and sodium hydroxide as needed.

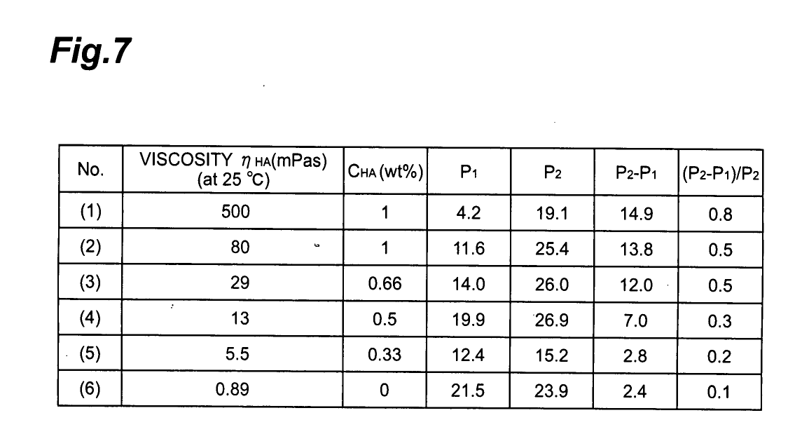

Five types of prefilled syringes containing solutions of the above compositions and differing viscosities (Specimen Nos. (1) to (5) in FIG. 7) and one type of prefilled syringe having a hyaluronic acid content of 0 wt % (Specimen No. (6) in FIG. 7) were prepared. The sodium hyaluronate in Specimen No. (1) had a molecular weight of 3,200,000, and the sodium hyaluronate in Specimen Nos. (2) to (5) had a molecular weight of 1,000,000.

FIG. 7 is a table showing the results obtained from measurements of the pushing forces P₁ and P₂, the pushing force difference P₂−P₁ and the difference ratio (P₂−P₁)/P₂ for the prefilled syringes of Specimen Nos. (1) to (6) in which hyaluronic acid solutions of differing viscosities (mPas) and mass percent concentrations CHA (wt %) were used.

Measurement was carried out using a precision universal testing machine (AG-IS autograph; manufactured by Shimadzu Corporation) as the compression test device. The load capacity was 1 kN, the load cell was 50 N, and the stroke velocity was 100 mm/min. The hypodermic needles used were 22G×1-½″ needles, and the measurement temperature was set to 25° C. The liquid medications were discharged from the tip of the hypodermic needle 14 into air.

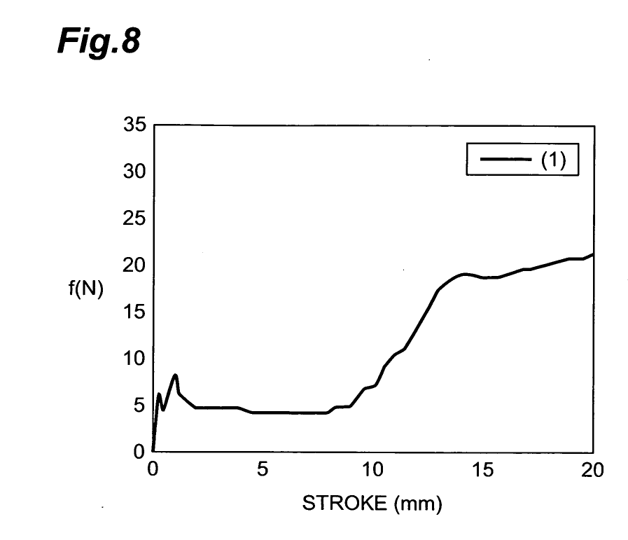

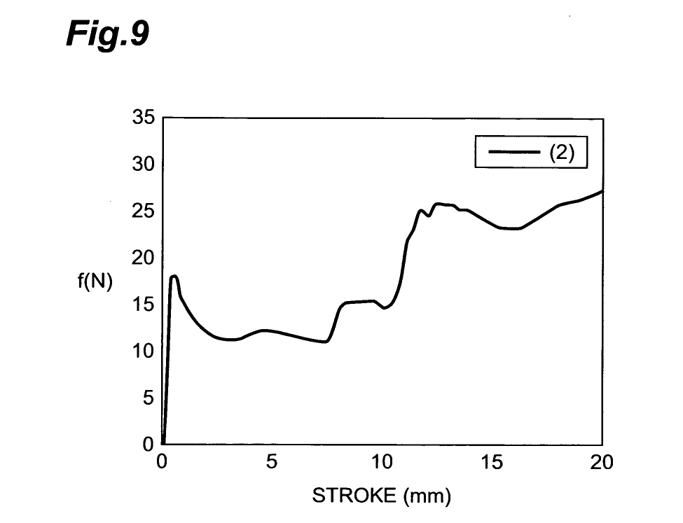

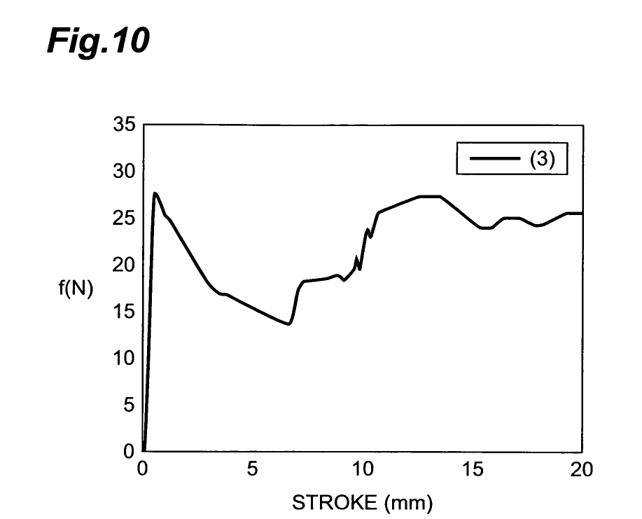

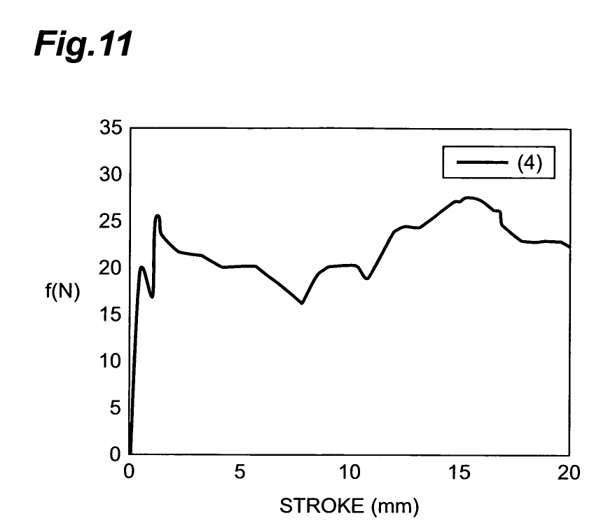

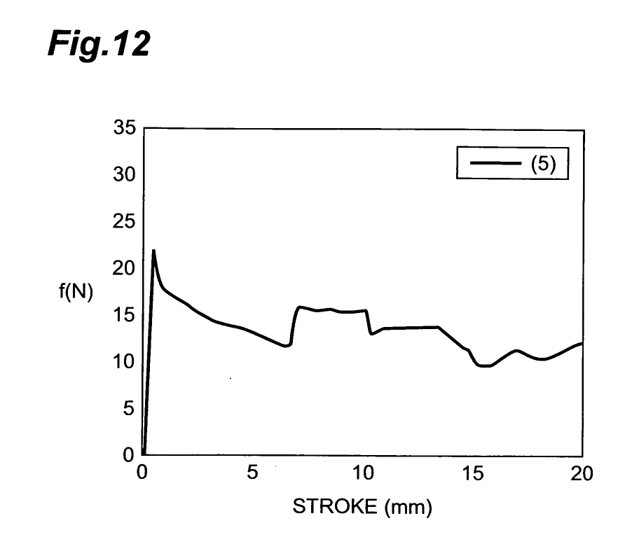

FIGS. 8 to 13 are graphs showing the relationship between stroke (mm) and f (N) in samples of Specimen Nos. (1) to (6).

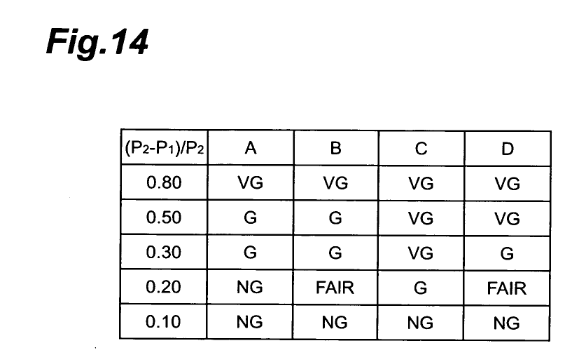

FIG. 14 shows the results obtained when four different operators A to D pushed the plungers. Concerning the symbols shown in the table (FIG. 14), “VG” indicates that the changeover between liquid medications was clearly perceived by sensing the above-described jolt, “G” indicates that the changeover was perceived, “FAIR” indicates that the changeover was difficult to perceive, and “NG” indicates that the changeover was not perceived.

All four operators were able to distinguish the changeover when the difference ratio (P₂−P₁)/P₂ was 0.3 or more. One of the operators clearly distinguished the changeover, and two others barely distinguished the changeover at a difference ratio (P₂−P₁)/P₂ of 0.2. When the viscosity η_{HA} was 10 mPas or more, that is, in Specimen Nos. (1) to (4), the difference ratio (P₂−P₁)/P₂ was larger than 0.2 or was 0.3 or more. Even at a difference ratio (P₂−P₁)/P₂ was 0.25, results identical to those for a ratio of 0.3 were obtained.

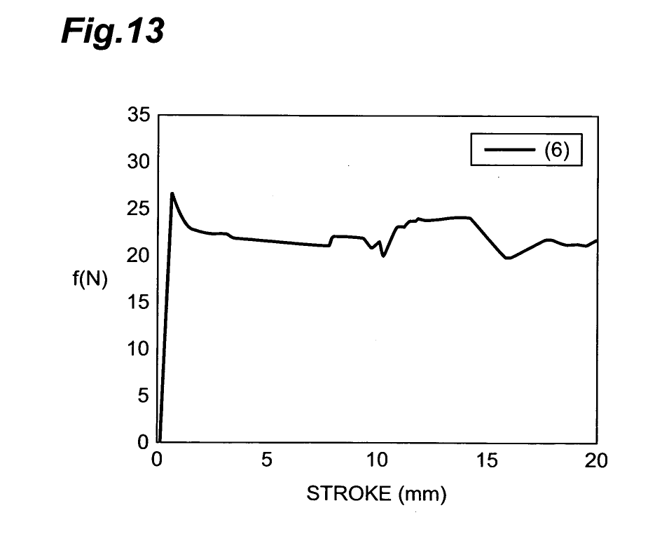

Referring to the graphs in FIGS. 8 to 12, in specimen Nos. (1) to (4), the f (N) changes substantially in the general region where the liquid medication changeover occurs near a stroke length of 10 mm. By contrast, in specimen No. (5), little change occurs. Referring to FIG. 13, in a case where a solution having substantially no viscosity was used (specimen (6)), substantially no change in f (N) was observed.

What is claimed is:

1. A prefilled syringe comprising:
a barrel;
an end stopper for sealing a first end of the barrel;
a middle stopper, interposed between the first end of the barrel and a second end of the barrel, for dividing the interior of the barrel into a front chamber and a back chamber;
a lidocaine solution enclosed within the front chamber; and
a hyaluronic acid solution enclosed within the back chamber,
wherein the viscosity η_{LD} (mPas) of the lidocaine solution at 25° C., the viscosity η_{HA} (mPas) of the hyaluronic acid solution at 25° C., the force P₁ (N) required to discharge the lidocaine solution enclosed within the front chamber from the second end of the barrel by pushing the end stopper toward the second end while causing the middle stopper to slide within the barrel and thereby applying dynamic frictional forces from an inner wall of the barrel to the end stopper and the middle stopper, and the force P₂ (N) required to discharge the hyaluronic acid solution enclosed within the back chamber from the second end of the barrel by pushing the end stopper toward the second end while applying a dynamic frictional force from the inner wall of the barrel to the end stopper satisfy the following conditions:
0.5≦η_{LD}≦5,
10≦η_{HA}≦600, and
(P₂−P₁)/P₂>0.2.

2. The prefilled syringe according to claim 1, wherein the mass percent concentration C_{LD} (wt %) of the lidocaine solution and the mass percent concentration C_{HA} (wt %) of the hyaluronic acid solution satisfy the following conditions:
0.5≦C_{LD}≦2, and
0.5≦C_{HA}≦2.

3. The prefilled syringe according to claim 1, which further satisfies the following conditions:
P₂−P₁>2, and
P₂<40.
